(19) United States
(12) Patent Application Publication
(10) Pub. No.: US 2001/0039296 A1
(43) Pub. Date: Nov. 8, 2001

(54) Method and composition for preventing or reducing the symptoms of menopause

(76) Inventors: Debasis Bagchi, Concord, CA (US); Gary Troxel, Lafayette, CA (US)

Correspondence Address: Oral Caglar, Esquire, Sheppard, Mullin, Richter & Hampton LLP, 48th Floor, 333 South Hope Street, Los Angeles, CA, 90071, US

(21) Appl. No.: 09/816,007

(22) Filed: Mar. 23, 2001

Related U.S. Application Data
(60) Provisional application No. 60/191,634, filed on Mar. 23, 2000.

(51) Int. Cl. A61K 35/78; A61K 31/05

(52) U.S. Cl. 514/734; 424/733; 424/734; 424/757

(57) ABSTRACT

A method for preventing and or reducing the symptoms of menopause in women comprises identifying women having or at risk for having such symptoms, and administering to them an effective amount of a composition comprising trans-resveratrol and other compounds that prevent or reduce the symptoms.

### BACKGROUND OF THE INVENTION

This application claims priority from U.S. Provisional Application Ser. No. 60/191,634. The invention relates generally to a method and composition for preventing or reducing the symptoms of menopause in women.

Menopause and its symptoms occur due to reduced production of estrogen in women. Estrogen is largely responsible for the changes that take place at puberty in girls, and it accounts for the secondary sexual characteristics of females. By direct action, estrogen causes growth and development of the vagina, uterus, and fallopian tubes. Estrogen acts in concert with other hormones to cause enlargement of the breasts through promotion of ductal growth, stromal development, and molding and shaping of body contours. With aging, and particularly beginning in the fifties for most women, the production of estrogen in the body begins to decline. This is a slow and gradual process that can continue for some years after menstruation has ceased. The menopausal symptoms are characterized by bone loss, osteoporosis, hot flashes, inappropriate sweating, vaginal dryness and/or itching, anemia and cardiovascular dysfunctions.

To reduce or prevent these symptoms, hormone replacement therapy or estrogen therapy is a common practice in women during menopause. Estrogenic activity is shared by many steroidal and nonsteroidal compounds. The most potent naturally-occurring estrogen in human beings is estradiol, followed by estrone and estriol. Women suffering from or at risk for suffering from these symptoms are administered estrogen in sufficient quantity to reduce or prevent them. However, administration of estrogens and synthetic therapeutic agents presents several problems, including increased incidence of breast and uterine cancers.

There remains a need for an effective method and composition for preventing and/or reducing the symptoms of menopause in a safe and convenient manner. The present invention fulfills this need and provides further related advantages.

### SUMMARY OF THE INVENTION

The present invention resides in a method to prevent and/or reduce the symptoms of menopause by administering a composition comprising an effective amount of trans-resveratrol in combination with one or more additional ingredients that serve to prevent or reduce these symptoms, to women identified as suffering from these health effects. The present invention is also embodied in a composition comprising trans-resveratrol and the additional ingredients. These ingredients include naturally occurring phytoestrogens and other compounds with specific beneficial effects. The composition can be administered in various forms, including pill, tablet, capsule, lozenge, gum, liquid or other commonly used forms of dietary supplements.

Other features and advantages of the present invention should become apparent from the following detailed description of the invention, taken with the accompanying drawings, which illustrate the principles of the invention.

### DETAILED DESCRIPTION OF THE INVENTION

The present invention resides in a method for preventing or reducing the symptoms of menopause in women by administering an effective amount of trans-resveratrol in combination with additional compounds that reduce menopausal symptoms. The present invention also resides in a composition comprising an effective amount of trans-resveratrol and one or more of these additional compounds.

Naturally occurring phytoestrogens are useful compounds in the present invention. These compounds can mimic the effects of estrogen in alleviating the negative health effects of menopause, without increased cancer risk. Trans-resveratrol is an antioxidant phytoestrogen compound present naturally mainly in the skin of grapes. One possible source of trans-resveratrol for use in the method or composition of the present invention is “Protykin”® brand extract, available from InterHealth Nutraceuticals of Benicia, Calif., an all-natural, high potency standardized extract of trans-resveratrol (50%) and emodin (<2%) derived from the dried rhizome of Polygonum cuspidatum. Trans-resveratrol is known to interact with estrogen receptors in the body, and a trans-resveratrol-estrogen receptor complex can help prevent osteoporosis, bone loss, hot flashes, and other menopausal symptoms.

Other naturally occurring phytoestrogens include soy derivatives, soy isoflavones, isoflavones from other plants, valerian root, and kava kava. For example, soy isoflavones, in particular diadzein and genistein, also have therapeutic properties as phytoestrogens. Soy isoflavones can prevent loss of bone mass and reduce hot flashes and mood swings associated with menopause.

Other compounds that may be administered with trans-resveratrol in the method and composition of the invention include calcium, vitamin D, zinc-L-monomethionine, vitamin B-12 and folic acid. Calcium lactate and vitamin D-3 have been demonstrated to increase bone mass to counteract possible bone loss. Zinc-L-monomethionine is known to promote immune function and sexual and reproductive characteristics, and also functions as an antioxidant Vitamin B-12 and folic acid are known to promote healthy cells and prevent anemia, a possible symptom of menopause.

These ingredients, in their various possible combinations, work synergistically with the trans-resveratrol in the composition to alleviate the various possible symptoms of menopause. The method involves consumption of the composition by the person, preferably as a regular treatment; e.g., daily. The composition contains an amount of trans-resveratrol and the other phytoestrogen or phytoestrogens sufficient to prevent and/or reduce the negative health effects of menopause. A preferred embodiment also can include other phytoestrogens not discussed above, or other compounds with therapeutic value to women suffering from symptoms of menopause. The composition also can include inert ingredients or diluents, such as sugar or other inert ingredients commonly used in food products. The composition may be in various forms commonly used for dietary supplements, including pill, tablet, capsule, lozenge, gum, liquid.

A preferred embodiment of the invention comprises administering a composition comprising 1-10 mg Protykin extract (as a source of 0.5 to 5 mg trans-resveratrol), 5-30 mg soy isoflavones, 25-150 mg calcium lactate, 40-400 IU vitamin D-3, 12.5-75 mg zinc-L-monomethionine, 3-10 μg vitamin B-12, and 5-20 μg folic acid. This particular composition comprises what are considered effective amounts of the various compounds, and it provides for particular synergistic effects for a wide range of symptoms associated with menopause and other female health remedies, such as the side effects associated with estrogen treatment therapies.

Although the invention has been disclosed in detail with reference only to the preferred embodiments, those skilled in the art will appreciate that additional methods and compositions can be made without departing from the scope of the invention.

We claim:

1. A method for preventing or reducing the symptoms of menopause in a person, the method comprising:
identifying a person suffering from or at risk for suffering from the symptoms; and
administering an effective amount of a composition comprising trans-resveratrol and other compounds that, when administered to the person, alleviate the symptoms.

2. The method of claim 1, wherein the other compounds are phytoestrogens.

3. The method of claim 2, wherein the phytoestrogens are soy derivatives, soy isoflavones, valerian root, or kava kava.

4. A composition comprising trans-resveratrol and other compounds that, when administered to a person in an effective amount, alleviate the symptoms of menopause.

5. The composition of claim 4, wherein the other compounds are phytoestrogens.

6. The composition of claim 5, wherein the phytoestrogens are soy derivatives, soy isoflavones, valerian root, or kava kava.

7. A composition comprising:
from about 0.5 to about 5 mg trans-resveratrol;
from about 5 to about 30 mg soy isoflavones;
from about 25 to about 150 mg calcium lactate;
from about 40 to about 400 IU vitamin D-3;
from about 12.5 to about 75 mg zinc-L-monomethionine;
from about 3 to about 10 μg vitamin B-12; and
from about 5 to about 20 μg folic acid.

8. A composition as described in claim 7, wherein the composition is in the form of a pill, tablet, capsule, lozenge, gum, or liquid.
